(19) United States
(12) Patent Application Publication
(10) Pub. No.: US 2004/0175368 A1
(43) Pub. Date: Sep. 9, 2004

(54) Use of crystal violet as photochemotherapeutic agent

(75) Inventor: Guilherme L. Indig, Madison, WI (US)

Correspondence Address: FOLEY & LARDNER, 150 EAST GILMAN STREET, P.O. BOX 1497, MADISON, WI, 53701-1497, US

(73) Assignee: Wisconsin Alumni Research Foundation

(21) Appl. No.: 10/751,302

(22) Filed: Jan. 2, 2004

Related U.S. Application Data
(63) Continuation of application No. 09/753,472, filed on Jan. 3, 2001, now abandoned.

(51) Int. Cl. A61K 31/13

(52) U.S. Cl. 424/93.7; 514/641

(57) ABSTRACT

Disclosed are compounds of the formula:

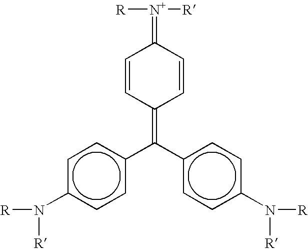

wherein R and R′ are independently hydrogen or C₁ to C₆ alkyl and the use of the compounds in the treatment of cancers and the purging of cancer cells from biological material to be transplanted into cancer patients.

### FIELD OF THE INVENTION

The invention is directed to a method of treating cancer using triarylmethane dyes as photochemotherapeutic agents. The invention is also directed to a method of purging cancerous cells from non-cancerous cells in autologous bone marrow grafts.

### BIBLIOGRAPHIC CITATIONS

Complete bibliographic citations to the references discussed herein are contained in the Bibliography section, directly preceding the claims.

### BACKGROUND AND DESCRIPTION OF THE RELATED ART

It is known that cancerous cells, such as tumor cells and leukemia cells, can be selectively purged from non-cancerous cells by photochemical methods. These methods are particularly useful in purging leukemia cells from a bone marrow graft before bone marrow transplantation. For instance, Merocyanine 540 (MC-540), a photosensitizing dye, has been used in photochemical purging of a patient's own (i.e., autologous) bone marrow graft. The effectiveness of MC-540-mediated photochemical purging, however, differs markedly in different leukemia cells lines.⁴⁶

Yamazaki and Sieber (1997)⁴⁵ found, however, that the selective lethality of MC-540 for leukemia cells could be synergistically increased by using MC-540 in conjunction with an alkyl-lysophospholipid, rac-2-methyl-1-octadecyl-glycero-(3)-phosphocholine (ET-18-OCH₃). These authors found that when photodynamic therapy (PDT) with MC-540 was followed by incubating the cells in ET-18-OCH₃, the MC-540-mediated photoinactivation of leukemia cells was synergistically enhanced, while the treatment only minimally reduced the survival of normal granulocyte-macrophage progenitors.

On the basis of a comprehensive investigation involving more than 200 cell lines/types of melanoma, adenocarcinoma, transitional cell carcinoma, squamous cell carcinoma, and normal epithelial cells, Chen has demonstrated that enhanced mitochondrial membrane potential is a prevalent cancer cell phenotype.¹ Only approximately 2% of all cells tested so far disobey this apparently dominant precept. Higher electric potentials have also been observed in the plasma membrane of a variety of carcinoma cells as compared to normal epithelial cells. Because cell and mitochondrial membrane potentials are negative inside, extensively conjugated cationic molecules displaying appropriate structural features can be electrophoretically driven through these membranes and accumulate into the cytosol and inside cell mitochondria. The mitochondrial membrane potential is typically more than 60 mV higher in carcinoma cells than in normal epithelial cells.^{1,2} As a result, a number of cationic dyes preferentially accumulate and are retained in a variety of tumor cells, presumably because the mitochondria of these cells are not capable of excreting the dyes with the same efficiency as normal cells.

The preferential uptake and retention of a variety of extensively conjugated cationic compounds by tumor cells have motivated the examination of mitochondrial targeting as a relevant therapeutic strategy for both chemotherapy and photochemotherapy of neoplastic diseases.^{3,7} However, the structural parameters that control the accumulation of these compounds into cell mitochondria are not entirely understood, and the lack of a robust model to describe the relationship between molecular structure and mitochondrial accumulation has prevented mitochondrial targeting from becoming a more dependable therapeutic strategy. Described herein is a method of treating cancer that utilizes cationic, triarylmethane dyes. While the invention is not limited to a particular mode of action, it is thought that the destruction of tumor cells wrought by the method arises via selective accumulation of the dye in the mitochondria of tumor cells.

Since 1953, when Nussenzweig⁸ first described the inactivation of the protozoan parasite Trypanosoma cruzi (the vector responsible for Chagas' disease) by the cationic triarylmethane dye crystal violet (CV⁺), this triarylmethane dye has been extensively used in blood banks in underdeveloped areas to prevent transfusion-associated transmission of Chagas' disease (American trypanosomiasis).⁹⁻¹⁴ CV⁺does not cause severe side effects in patients who receive blood treated with it, nor are the functions of blood cells jeopardized as a result of the chemoprophylaxis. ^{12,14} The safety of CV⁺is further demonstrated by its use as an anthelmintic, an antiseptic in umbilical cords of newborns and burn patients, and a colorant in food and cosmetics.^{11,15,16} The trypanocidal activity of CV⁺is known to develop at the mitochondrial level,¹⁰ and because it has been demonstrated that light enhances the trypanocidal effects of this triarylmethane (TAM⁺) dye,⁹⁻¹⁴ it was thought that CV⁺might be a candidate for use in photodynamic therapies to kill cancer cells selectively, and/or to inhibit the growth, spread, and proliferation of cancer cells.

### SUMMARY OF THE INVENTION

A first embodiment of the invention is directed to a method of killing cancer cells or inhibiting growth of cancer cells, in vitro, in vivo, or ex vivo. The method comprises first contacting the cancer cells with a compound selected from the group consisting of:

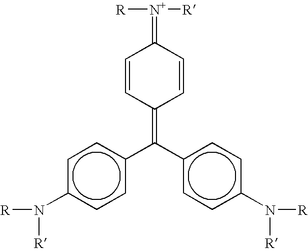

wherein each R and R′ is independently selected from the group consisting of hydrogen and C₁-C₆ linear or branched alkyl. Then, the cancer cells so treated are exposed to radiation of a suitable wavelength to photoactivate the compound, whereby cancer cell death or cancer cell growth inhibition results. The method can be used to treat solid neoplastic tumors and circulating neoplasms such as leukemia and the like.

A second embodiment of the invention is directed to a method of purging malignant cells from a mixture containing malignant and non-malignant cells. The method comprises contacting the mixture with a compound selected from the group consisting of:

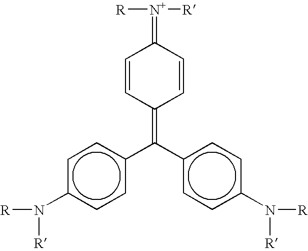

wherein each R and R′ is as described above. The mixture so treated is then exposed to radiation of a suitable wavelength to photoactivate the compound, thereby inducing death of malignant cells in the mixture.

This embodiment of the invention is preferred to be used in preparing autologous bone marrow transplants for reimplantation into the subject from which the transplant was taken. The subject will normally be a mammal (human or other mamal) suffering from a neoplasm involving the cells found in bone marrow, such as leukemia.

It has been found that crystal violet (CV⁺) exhibits pronounced phototoxicity toward leukemia cells and low toxicity toward normal hematopoietic cells. On the basis of the selectivity with which the phototoxic effect of CV⁺develops toward tumor cells as compared to normal cells, the principal advantage and benefit of the invention is that this triarylmethane dye, and closely-related homologs, can be used in photodynamic therapy to destroy and/or inhibit the growth of cancer cells, while leaving non-cancerous cells viable. A primary use of the invention, therefore, is a novel purging protocol to promote the elimination of residual tumor cells from autologous bone marrow grafts with minimum toxicity toward normal cells.

### BRIEF DESCRIPTION OF THE DRAWINGS

FIG. 1 depicts simultaneous two-photon laser microscopy images of dye-loaded RBL cells excited at 750 mm. From left, fluorescence from CV⁺(600 nm to 660 nm), Rh123 (520 nm to 550 nm), and NAD(P)H (400 nm to 500 nm). RBL cells incubated for 10 minutes (upper row) or 60 minutes (lower row) with 0.2 μM CV⁺and subsequently for 30 minutes with 0.05 μM Rh123.

FIG. 2 depicts two-photon laser microscopy images of RBL cells loaded with EV⁺. Fluorescence images obtained after 10 minutes (left panel) and 60 minutes (right panel) of EV⁺incubation. Emission measured in the 600 to 660 nm region.

FIG. 3 shows the photoinactivation of L1210 leukemia cells (upper panel) and murine CFU-GM cells (lower panel) sensitized by TAM⁺dyes. Data points represent mean colony counts ± standard errors of four replicate culture dishes. Untreated leukemia cells (0 min) generated a mean number of 134.5 colonies per 400 cells plated. Untreated bone marrow cells (0 min) generated a mean number of 482.5 granulocyte-macrophage colonies per 500,000 nucleated cells plated. Cells incubated for 60 minutes with TAM⁺1.0×10⁶ M. Fluence rate=27 W/m².

FIG. 4 illustrates routes of deactivation and photobleaching of CV⁺. Other, parallel reactions, may also take place.

FIG. 5 shows transient decay curves for air-equilibrated samples of CV⁺non-covalently bound to BSA as a monomer (M; {BSA}=40 μM) and as a dimer (D; {BSA}=2.5 μM). Phosphate buffer 10 mM, pH 7.3; T=25° C.; {CV}=10 μM.

### DETAILED DESCRIPTION OF THE INVENTION

Extensively conjugated cationic molecules with appropriate structural features will accumulate into the mitochondria of living cells, a phenomenon typically more prominent in tumor cells than in normal cells. It has now been found that a variety of tumor cells also retain pertinent cationic structures for longer periods of time compared to normal cells. While not being bound to a particular mode of action, the present method utilizes mitochondrial targeting as a selective therapeutic strategy of relevance for both chemotherapy and photochemotherapy of neoplastic diseases in general, and leukemia in particular.

The present invention is directed to the use of triarylmethane (TAM⁺) dyes, the preferred dye being crystal violet (CV⁺) for photochemotherapy of neoplastic conditions. CV⁺stains neoplastic cell mitochondria with efficiency and selectivity. Upon exposure to suitable wavelengths of energy, this dye (and closely-related homologs) exhibits pronounced phototoxicity toward neoplastic cells. As illustrated in the Examples that follow, CV⁺exhibits pronounced phototoxicity toward L1210 leukemia cells but comparatively small toxic effects toward normal hematopoietic cells (murine granulocyte-macrophage progenitors, CFU-GM cells). On the basis of a comparative examination of chemical, photochemical, and phototoxic properties of crystal violet and other triarylmethane dyes, certain interdependencies between molecular structure and selective phototoxicity toward tumor cells have been identified. These structure-activity relationships provide useful guidelines for the novel purging protocols described herein, protocols that selectively eliminate residual tumor cells from autologous bone marrow grafts with minimum toxicity to normal hematopoietic stem cells.

The following experiments are provided for illustrative purposes only, in an effort to describe the claimed invention clearly and completely. It is understood that the experiments described below do not limit the invention claimed herein in any fashion.

Chemicals:

Chlorine salts of the triarylmethane dyes ethyl violet (EV⁺), victoria blue R (VBR⁺), victoria pure blue BO (VPBBO⁺) from Aldrich Chemical (Milwaukee, Wis.), and CV⁺from Sigma (St. Louis, Mo.) were recrystallized from methanol and dried under vacuum. The purity of recrystallized TAM⁺dyes was assessed by thin-layer chromatography (TLC, silica gel, methanol-acetic acid 95:5, vol/vol). N-2-hydroxyethyl piperazine-N′-2-ethanesulfonic acid (HEPES) was obtained from Research Organics (Cleveland, Ohio); methylcellulose (4000 cPs) from Fluka Chemical; recombinant granulocyte/macrophage colony stimulating factor (GM-CSF; murine sequence) from R&D Systems (Minneapolis, Minn.); bovine serum albumin (BSA), fetal bovine serum (FBS), and alpha-modified Dulbecco's medium (alpha-medium) from Sigma; and minimal essential media and newborn calf serum (NCS) from Gibco BRL. Rhodamine 123 (Rh123) from Molecular Probes and 1-octanol from Aldrich were used as supplied. Water was distilled, deionized, and filtered before use (Millipore Milli-Q system; resistivity, 18 MΩ cm). The characterization of reaction photoproducts was carried out by electronic spectroscopy and TLC as described elsewhere.¹⁷

Spectroscopic and Photochemical Investigations in Solution:

Spectrophotometric studies were performed with a Shimadzu UV-2101PC spectrophotometer. 1-Octanol/water partition coefficients¹⁸ (P), were determined at 25° C. using equal volumes of water and 1-octanol. Typically, five distinct solutions of each dye in the concentration range between 1 μM and 10 μM were prepared in deionized water and subsequently equilibrated with 1-octanol. After the equilibrium was reached, the final dye concentrations in the aqueous and/or organic phases were determined by absorption spectroscopy. For the measurement of photobleaching quantum efficiencies, the samples were placed in standard 10-mm (optical path) quartz cells at a distance of approximately 10 cm from the light source and photolyzed using the 532-nm line of an Nd:YAG laser model 7010 from Continuum operating at a repetition rate of 10 Hz. The defocused laser beam (circular profile with a diameter of about 5 mm) was directed to the center of the quartz cell. The absolute photolysis energy (or the number of 532-nm photons per laser pulse) was kept constant over the course of any specific experiment. The temperature-controlled cell holder allowed continuous magnetic stirring of the samples during photolysis. The photobleaching quantum efficiencies were based on the photobleaching efficiency of a classical chemical actinometer, potassium ferrioxalate, ^{19,20} and determined considering only the first 5 to 10% decrease in dye concentration. The photolysis energy was adjusted to appropriate levels with the use of a calibrated solid-state joulemeter model PM30VI from Molectron.

The laser flash photolysis equipment used in the Examples is similar to a previously-described system.²¹ The major assembly components are a nanosecond dye laser (Continuum, ND6000) pumped by an Nd:YAG laser (Continuum, 7010), used as the excitation light source; a 300 W xenon arc lamp system (Oriel, 66084), which provides the analysis beam; a monochromator (CVI, CM110); a red-sensitive photomultiplier tube (Hamamatsu, R446); and a dual-channel 600-MHz digital oscilloscope (LeCroy, 9360).

Cells:

L1210 murine leukemia cells (CCL 219; American Type Culture Collection, Manassas, Va.) were cultured in alpha-medium supplemented with 10% FBS, incubated in a humidified atmosphere of 5% CO₂ in air and harvested in exponential growth phase. Rat basophilic leukemia (RBL) cells were plated at a density of 2.3×10⁵ cells per 2 mL of growth media (minimal essential media (GibcoBRL) supplemented with 10% FBS and 10% NCS) on observation dishes with a glass coverslip bottom and allowed to adhere overnight at 37° C. Female C57BL/6J×DBA/2J mice (approximately 6 months old; The Jackson Laboratory, Bar Harbor, Me.) served as a source of normal bone marrow cells.

Two-Photon Microscopy:

RBL cells were incubated in the dark at a concentration of 1.1×10⁵ cells/mL for periods ranging from 1 minute to 1 hour with fresh growth media containing 0.2 μM CV⁺or Ev⁺and subsequently incubated for 30 minutes in the dark with fresh growth media containing 0.05 μM Rh123; After a final cycle of washing and incubation for 15 minutes in fresh growth media, the media were replaced by modified Tyrode's solution containing 5 mM glucose and bovine serum albumin, and the cells were subjected to two-photon imaging. The examination of TAM⁺accumulation into mitochondria was carried out through the comparison of the spatial distribution of the fluorescence of these dyes in the cellular environment with that of Rh123 (a classical mitochondrial marker^{1,3}) and the endogenous NAD(P)H. Fluorescence distributions within living RBL cells were obtained with a laser scanning multiphoton microscope (Bio-Rad MP1024). Pulsed (75 fs, 80 Mhz) infrared 750-nm laser light was focused to a diffraction-limited spot with a Zeiss F-Fluar oil immersion objective (NA=1.3) and raster-scanned across the sample. Three external detectors located in the Fourier plane of the microscope and equipped with appropriate sets of longpass dichroic and bandpass filters (from Chroma) were used for the simultaneous detection of two-photon excited fluorescence of NAD(P)H (400-500 nm), Rh123 (520-550 nm), and CV⁺or EV⁺(600-660 nm). To obtain sufficient fluorescence from CV⁺,EV⁺, and cellular NAD(P)H autofluorescence (all inefficient fluorophores), average excitation powers of approximately 18 mW were required. A detailed description of the experimental setting used in two-photon microscopy is known in the art.²²

Uptake of Crystal Violet by L1210 Cells:

L1210 cells were suspended at a concentration of 1.0×10⁶ cells/mL in HEPES-buffered (10 mM, pH 7.4) alpha-medium supplemented with FBS (12%), and the TAM⁺dyes were added from 1.0×10⁻⁴M stock solutions in 50% ethanol to final concentrations of 1.0×10⁶M. The cells were subsequently incubated in the dark for 1 hour at 37° C., pelleted, washed with dye-free medium, and extracted with ethanol (1 mL per 5×10⁻⁶ cells). The dye content of each extract was determined spectrophotometrically using calibration curves assembled using suitable dilutions of authentic dye in ethanol extracts of cells that had been incubated in dye-free medium. Control experiments demonstrated that under these experimental conditions (1.0×10⁻⁶ cells/mL, 12% FBS) and within the time frames when the measurements were taken, TAM⁺dyes at a final concentration of 1.0×10⁻⁶ M showed negligible or no dark toxicity toward L1210 cells.

Dye-Sensitized Photoinactivation of Cells:

The cells were incubated for 60 minutes in the presence of the TAM⁺dyes as described above, washed once with dye-free HEPES-buffered alpha-medium supplemented with 5% FBS, and resuspended at a density of 10⁶ cells/mL in HEPES-buffered alpha-medium supplemented with 12% FBS. Capped clear polystyrene tubes (15 mL) containing the cell suspension (2 mL) were mounted on a Plexiglas disk that rotated at approximately 60 rpm between two banks of tubular fluorescent lights (five lights per bank; F20T12.CW; General Electric, Cleveland, Ohio)²³ and irradiated for up to 90 minutes. The fluence rate at the sample site was 27 W/m², as determined by a model S351A power meter (United Detector Technology, Hawthorne, Calif.) equipped with a model 262 detector and radiometric filter number 1158. For select experiments, the cell suspensions were treated with oxygen or argon immediately before the photoirradiation period as previously described.²⁴ After irradiation, the cells were washed twice with dye-free HEPES-buffered alpha-medium supplemented with 5% FBS, and all subsequent manipulations were performed in the dark or under low level ambient light.

In Vitro Cl nal Assays:

The survival of photoinactivated and untreated L1210 leukemia cells was assessed using an in vitro clonal assay as described elsewhere. 2 CFU-GM cells were assayed as previously described.^{26,27} Cells that had been exposed to dye but not to light, to light but not to dye, or to neither dye nor light, served as controls.

Intracellular Distribution of CV⁺and EV⁺in Living Cells:

Two-photon laser scanning microscopy^{22,28} was used to obtain information on intracellular distribution and mitochondrial accumulation of CV⁺and EV⁺in RBL cells. The experimental strategy used a mitochondrial marker (Rh123) and cellular NAD(P)H autofluorescence²⁹ to probe the cellular distribution of TAM⁺dyes and to characterize early alterations in mitochondria structure and bioenergetics associated with the cytotoxic effect of these compounds. Simultaneous two-photon images of NAD(P)H, Rh123, and TAM⁺fluorescence were obtained by exciting RBL cells at 750 nm with a Ti:Sapphire laser (75 fs, 80 MHz, 18 mW) as the excitation source. No morphologic alterations or induction of fluorescence attributed to cellular photodamage was observed during the brief imaging period (approximately 3 seconds), and the bleaching of the three fluorophores was also negligible during this period. The cell fluorescence was analyzed in three detection channels for the simultaneous characterization of the spatial distribution of each fluorophore of interest, NAD(P)H, Rh123, and TAM⁺, inside the RBL cells.

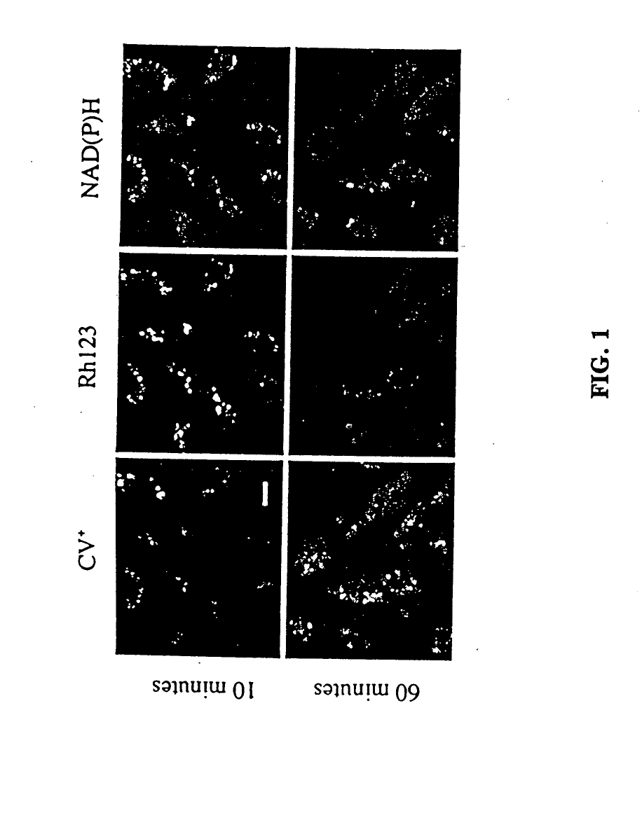

The images obtained in the control experiments without TAM⁺and Rh123 displayed bright punctate NAD(P)H autofluorescence distribution. This distribution was analogous to the distribution observed for mitochondria stained only with Rh123, but with the addition of a background autofluorescence distribution throughout the cytoplasm and to a lesser degree in the nucleus. In the case of cells incubated for 10 minutes in the presence of CV⁺and subsequently with Rh123, the fluorescence associated with CV⁺, Rh123, and NAD(P)H had similar perinuclear distributions in the cell, which is consistent with mitochondrial localization ^{1,29} (see FIG. 1). Rh123 is known to stain mitochondria with remarkable efficiency and selectivity. For this particular dye, the membrane potential-driven contribution of the mitochondrial accumulation phenomenon is clearly the dominant contribution. Accordingly, on depolarization of the mitochondrial membrane of living cells, Rh123 no longer accumulates or is retained in the mitochondria.^{1,2} After 1 hour of CV⁺incubation, Rh123 was no longer efficiently retained by RBL cell mitochondria, the mitochondria appeared swollen (approximately 2-3 μm in diameter), and cellular autofluorescence was less intense and less punctate (FIG. 1). This observation indicates that the mitochondrial inner membrane potential has been reduced and NADH has been oxidized to NAD⁺. The approach of monitoring NAD(P)H fluorescence in living cells for the assessment of mitochondrial bioenergetics was originally introduced by Chance.²⁹ In model studies carried out with isolated rat liver mitochondria, the depolarization of the mitochondrial membrane by CV⁺has been attributed to uncoupling³⁰ and induction of mitochondrial permeability transition.³¹

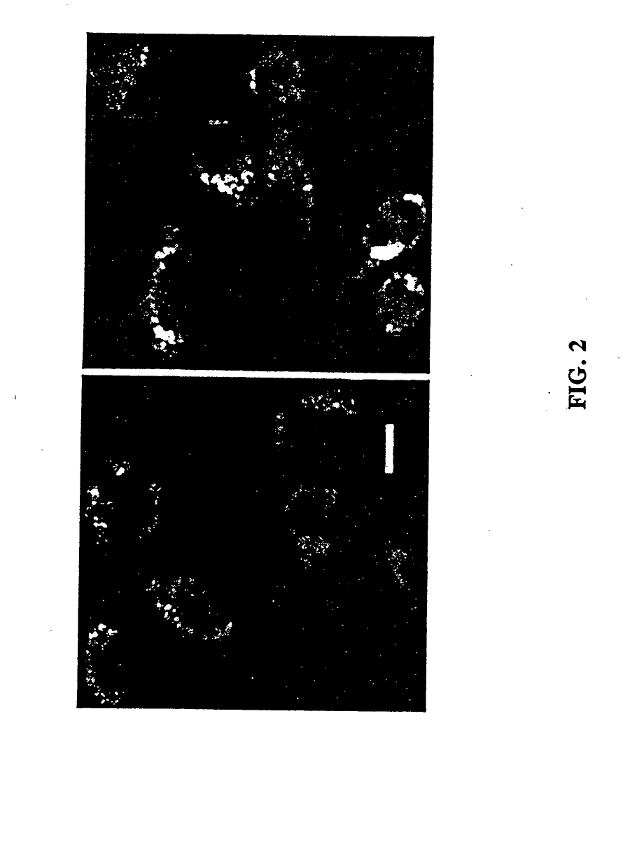

When EV⁺was used in place of CV⁺, similar mitochondrial effects were observed. The comparison of the fluorescence patterns of EV⁺, Rh123, and NAD(P)H after 10 minutes of RBL incubation with EV⁺indicated that this dye also accumulates into cell mitochondria. However, significant EV⁺fluorescence background was observed throughout the cytoplasm, suggesting that a substantial fraction of the EV⁺molecules taken up by RBL cells may also localize in other subcellular compartments (see FIG. 2, left panel). In addition, after depolarization of the mitochondrial membrane and release of Rh123, substantial EV⁺fluorescence was still observed in mitochondrial regions (1 hour incubation, right panel of FIG. 2).

For extensively conjugated cationic structures displaying appropriate lipophilic/hydrophilic character, the contribution of membrane partitioning on the mechanism of dye accumulation into the cytosol and inside cell mitochondria may be negligible. In these cases, mitochondrial accumulation appears to be controlled primarily by membrane potential-driven electrophoresis and chemical potentials, as described by the Nernst equation.² On increasing the lipophilic character of the conjugated cationic structure, a higher contribution from the partitioning phenomena is expected to occur, and consequently mitochondrial accumulation may be preserved even after the mitochondrial membrane is depolarized. For highly lipophilic compounds, membrane partitioning can be expected to represent the dominant contribution. A higher contribution of membrane partitioning phenomena on the mechanism of mitochondrial accumulation of EV⁺, compared with CV⁺, was indicated by the fact that substantial EV⁺fluorescence was still observed in mitochondrial regions after depolarization of the mitochondrial membrane.

Phototoxic Effects of TAM⁺Dyes Toward Leukemia and Normal Hematopoietic Cells:

To explore whether CV⁺, EV⁺, and other structurally related TAM⁺dyes can promote the selective destruction of tumor cells with minimum toxicity toward normal cells, a model preclinical study was conducted in which the phototoxicity of TAM⁺dyes toward leukemia (L1210) and normal hematopoietic (murine CFU-GM) cells was compared under experimental conditions in which the respective thermal (dark) toxicity was small for both cell lines (1.0×10⁻⁶ M dye, 1.0×10⁶ cells/mL; 12% FBS). The choice of murine CFU-GM cells to assess the toxicity to normal hematopoietic cells was motivated by the fact that they are relatively frequent in bone marrow (thus allowing depletions over two to three orders of magnitude to be documented) and also because, in purging applications, a good correlation is often found between the preservation of CFU-GM cells and the recovery of the neutrophil compartment. For certain purging agents, the survival of CFU-GM cells has also proven to be a reliable indicator of the radioprotective capacity of purged marrow.²⁶

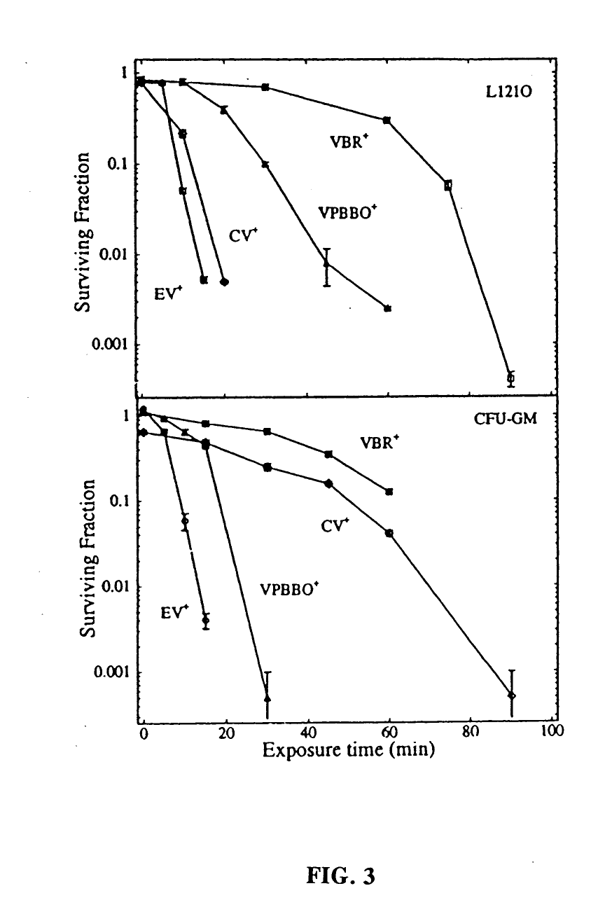

FIG. 3 shows the efficiency of a series of TAM⁺dyes to photoinactivate L1210 leukemia cells and CFU-GM cells as a function of time of light exposure. The comparison between the data shown in the upper (L1210) and lower (CFU-GM) panels of FIG. 3 clearly indicates that from among the dyes tested in this Example, CV⁺is the preferred TAM⁺dye for photodynamic therapy due to its ability to destroy cancer cells selectively. CV⁺is the preferred compound for use in the subject method because it has been shown to be substantially more efficient in selectively photoinactivating L1210 cells in the presence of CFU-GM cells. After relatively short periods of light exposure (c.a. 20 minutes), the surviving fractions of L1210 cells represented only 0.3 to 0.4% of their initial values, whereas in the case of CFU-GM cells the respective surviving fractions were still in the range of 60 to 70% of their initial values.

The phototoxic effects of the other TAM⁺dyes tested do not exhibit the same marked selectivity toward leukemia cells as does CV⁺. VBR⁺displays the lowest efficacy in destroying both L1210 and CPU-GM cells of any of the compounds tested, VPBBO+displays intermediate efficacy, and EV⁺is phototoxic toward both cell lines (FIG. 3). Interestingly, VPBBO⁺is a TAM⁺dye that has received some attention as a potential photosensitizer for PDT, despite the fact that it displays the worst degree of tumor cell selectivity.^{6,32,23}

Although VPBBO⁺mediates the photoinactivation of L1210 leukemia cells with reasonable efficiency, this dye was also found to be considerably more efficient in inactivating normal hematopoietic cells. In contrast, CV⁺, the preferred compound for use in the present invention (and a TAM⁺dye successfully used in tropical medicine for several decades^{8,13,14,34}) has never been considered for use in photodynamic therapy. As noted above, the previously unrecognized ability of CV⁺to kill tumor cells upon exposure to radiation of the proper wavelength, with efficacy and selectivity, makes CV⁺the preferred TAM⁺dye for use in the subject invention.

Correlation Between Phototoxic Effects and Structural Characteristics of TAM⁺Dyes:

The model preclinical study on the phototoxicity of TAM⁺dyes toward leukemia and normal hematopoietic cells has revealed CV⁺as a drug whose structural characteristics are well suited for the promotion of selective photochemical destruction of residual tumor cells in autologous bone marrow grafts with comparatively low toxicity toward normal cells. In an effort to determine the structural features of CV⁺that give rise to this selectively and to identify structural guidelines for developing still more effective mitochondrial-targeted anti-tumor drugs, a systematic study was conducted on how molecular structure affects chemical and photochemical properties of TAM⁺dyes, and how these properties modulate both selectivity and phototoxicity.

Photoreactivity Versus Observed Toxicity:

The extent to which a specific TAM⁺dye is capable of inducing specific destruction of residual tumor cells in bone marrow grafts depends primarily on its preferential uptake by tumor cells compared with normal cells, the site(s) of subcellular localization, and the quantum efficiency of the photochemical events that lead to the lethal toxic effects. TAM⁺dyes show very short singlet lifetimes in low viscosity media because of fast, nonradiative relaxation processes that occur via rotational motions of their aromatic rings.^{17,35,36} When free in aqueous media, the photoreactivity of TAM⁺dyes is extremely poor, and no significant phototoxicity should be expected from these dyes under such circumstances. However, in aqueous media, TAM⁺dyes bind efficiently to a variety of biopolymer polyelectrolytes through noncovalent interactions, including proteins and nucleic acids.^{15,17,37}

Accordingly, in complex biologic systems, these photosensitizers are not expected to be found free in solution to any significant extent, but rather bound to biopolymers and supramolecular structures. When TAM⁺molecules are located in binding micro-environments that render steric hindrance to the rotational motions of their aromatic rings, the efficiency of nonradiative relaxation processes decreases compared with dye molecules free in aqueous media. As a result, fluorescence and intersystem crossing become more competitive events, and photoreactivity tends to increase.^{17,37}

To explore how the photoreactivity of the TAM⁺dyes in complex biologic environments correlates with the observed phototoxic effects toward tumor and normal cells, the photo-bleaching efficiency of TAM⁺dyes noncovalently bound to a model biologic host (bovine serum albumin, BSA) was measured. For the case of CFU-GM cells, the efficacy of photoinduced cell destruction mediated by TAM⁺dyes precisely paralleled the photochemical reactivity of these photosensitizers. Both, phototoxicity (FIG. 3, lower panel) and photoreactivity (Table 1) followed the decreasing order EV⁺>VPBBO⁺>CV⁺>VBR⁺. However, the toxic effect of CV⁺toward L1210 leukemia cells was substantially higher than what would be expected solely on the basis of photochemical considerations. In fact, the phototoxicity of CV⁺toward L1210 cells was comparable to that observed for the case of the most photoreactive triarylmethane tested, EV⁺(FIG. 3, upper panel).

Phototoxicity Versus Cellular Uptake:

Data on cellular uptake (Table 1) for the dyes tested herein indicated that CV⁺is the dye most efficiently taken up by L1210 cells. Thus, enhanced tumor cell uptake is thought to be a major mechanistic mode of action resulting in observed behavior of CV⁺, and provides a plausible explanation for the fact that the phototoxic effect of CV⁺toward L1210 cells does not follow the trend predicted by the relative photoreactivity along the TAM⁺dye series. For the other TAM⁺dyes tested to date, EV⁺, VPBBO⁺, and VBR⁺, both the efficiency of cellular uptake by L1210 cells and dye photoreactivity paralleled phototoxicity.

TABLE 1
Photobleaching Efficiencies, Partition Coefficients (P),
and Cellular Uptake Values for TAM⁺ Dyes

| | | | Dye Uptake by |
|---|---|---|---|
| | Photobleaching | | L1210 Cells |
| | Efficiency^{a} | | (×10⁻⁷ |
| | (×10⁵) | (P) | Molecules/Cell) |
| EV⁺ | 3.3 | 237 | 17.6 |
| VPBBO+ | 3.0 | 180 | 11.6 |
| CV⁺ | 1.3 | 2.4 | 21.4 |
| VBR+ | 0.5 | 39 | 8.8 |

^{a}10 mM buffer pH 7.3.
{Dye} = 10 μM;
{BSA} = 40 μM

Lipophilic/Hydrophilic Character and Cellular Uptake:

With the exception of CV⁺, the relative efficiency by which L1210 cells take up TAM⁺molecules correlates with the lipophilic/hydrophilic character of these compounds, as assessed through the measurement of 1-octanol/water partition coefficients (Table 1). For VBR⁺, VPBBO⁺, and EV⁺, the higher the partition coefficient, the higher the cellular accumulation. However, the dye showing the lowest 1-octanol/water partition coefficient (P) among the TAM⁺dyes considered herein, CV⁺(P =2.4), is the dye most efficiently taken up by L1210 cells. The enhanced L1210 cellular uptake observed for the case of CV⁺, compared with the other more lipophilic TAM⁺structures, is in keeping with the hypothesis that the cellular uptake and mitochondrial accumulation and retention of a cationic compound displaying appropriate structural features can be primarily driven by membrane potentials rather than by the partitioning phenomena. In the case of the model mitochondrial dye Rh123, whose cellular uptake and mitochondrial accumulation is known to be driven almost exclusively by membrane potentials,^{1,2} the 1-octanol/water partition coefficient is only 0.24 as measured under exactly the same conditions as those used for the TAM⁺dyes. Therefore, because the values of partition coefficients of CV⁺and Rh123 are relatively close, it is reasonable to presume that the enhanced CV⁺uptake by L1210 cells is a direct consequence of a more appropriate lipophilic/hydrophilic character of this dye, as compared to the other TAM⁺structures considered here. The high values of partition coefficients of VBR⁺(39), VPBBO⁺(180), and EV⁺(237) suggest that for these dyes, the membrane partitioning phenomena must represent a much more pronounced contribution to the respective mechanisms of subcellular distribution and mitochondrial accumulation. Indeed, the two-photon laser microscopy data suggested that mitochondrial membrane partitioning plays a more prominent role in the mitochondrial localization and accumulation of EV⁺than it does for CV⁺.

Dye Structure Versus Selective Toxicity:

Considering that enhanced mitochondrial membrane potential is a prevalent cancer cell phenotype,^{1,5} it can be inferred that the accumulation of CV⁺into tumor cells and respective mitochondria will be typically greater than its accumulation into normal hematopoietic stem cells, in agreement with the substantial differences observed in the efficacy of photoinactivation of L1210 cells and murine CFU-GM cells mediated by this compound (see FIG. 3). Because CV⁺shows a slightly higher lipophilic character than Rh123, the mechanism of mitochondrial accumulation of CV⁺is expected to show a slightly more prominent dependence on membrane partitioning compared to Rh123. A higher contribution from membrane partitioning on the mechanism of mitochondrial accumulation of CV⁺as compared to Rh123 was indicated by the two-photon imaging experiments. A modest CV⁺fluorescence was still observed in the mitochondria of RBL cells after depolarization of the mitochondrial membrane (FIG. 1).

The difference between the partition coefficient of CV⁺and Rh123 is seemingly not large enough to disrupt the selective nature of the phototoxic effect of CV⁺toward tumor cells. However, such selectivity is lost on further increasing the lipophilic character of TAM⁺structures. Therefore, one hypothesis is that the level of selectivity to which TAM⁺dyes can mediate the destruction of tumor cells in contrast to normal cells fundamentally depends on the lipophilic/hydrophilic character of the molecular structure of each specific compound. For the more lipophilic molecules, the contribution of lipophilic partitioning on the mechanism of cellular uptake and subcellular distribution is high enough to bring the loading of different cell types to comparable levels, thereby precluding selectivity toward tumor cells from taking place. For the more lipophilic cationic dyes the mitochondrial targeting is presumably less specific, as suggested by the extensive background fluorescence observed for the case of EV⁺throughout the RBL cells (FIG. 2). Under conditions of equivalent loading and when the targeting is less specific, L1210 cells are apparently more resistant to the phototoxic effects of TAM⁺dyes than CFU-GM cells. This is indicated by the observation that the phototoxic effect of EV⁺, VBR⁺, and VPBBO⁺is either slightly (VBR⁺and EV⁺) or considerably (VPBBO⁺) more prominent toward normal cells as compared to tumor cells (FIG. 3).

Based on the known properties of Rh123, we hypothesize that the closer the lipophilic/hydrophobic character of a particular TAM⁺molecule is to that of Rh123, the higher its specificity as a mitochondrial-targeting drug and the better its selectivity toward tumor as compared to normal cells. The comparison of the phototoxicity profiles of TAM⁺dyes toward L1210 leukemia and normal hematopoietic cells has indicated that CV⁺is the best and preferred TAM⁺dye tested that displays appropriate therapeutic properties.

The Photochemical Mechanism of Phototoxicity:

The nature of the spectral changes observed on 532-nm laser photolysis of BSA-bound TAM⁺dyes was highly conserved among the dyes tested, suggesting that the mechanism of photobleaching was also conserved among the series. Through ultraviolet-visible spectroscopy and TLC analysis, the formation of both benzophenone-type photoproducts and reduced (leuco) forms of the TAM⁺dyes in irradiated samples of BSA-TAM⁺omplexes has been characterized.

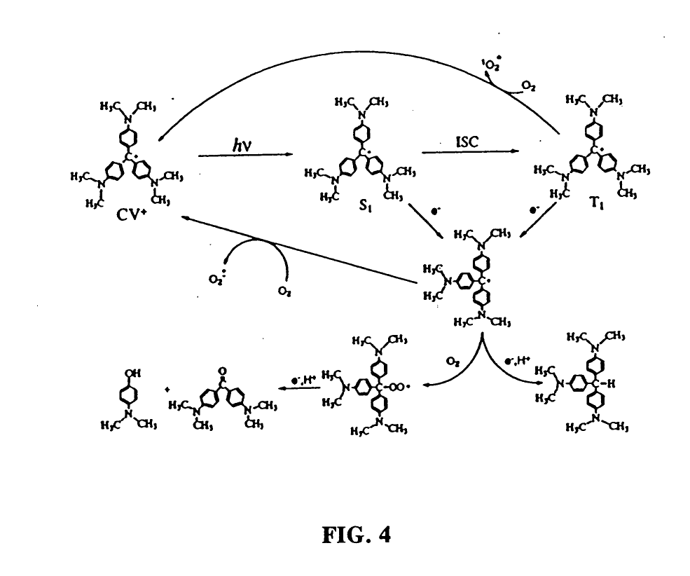

The reduction of a TAM⁺dye to its leuco derivative is a two electron process (formally H+e³¹ or 2 e⁻+H⁺) initiated, in the case of BSA-TAM⁺complexes, by a photo-induced electron or hydrogen atom transfer from the protein to the dye moiety.¹⁷ After initiation, and following the reaction coordinates of the bleaching process, the semi-reduced carbon-centered dye radical can either react with dissolved molecular oxygen to produce the benzophenone-type photoproduct or accommodate a second electron to form the leuco derivative of the TAM⁺dye. These reaction pathways are illustrated in FIG. 4 using CV⁺as an example. The photochemistry of BSA-CV⁺complexes has been previously investigated in great detail.^{17,37} For this complex, Michler's ketone and leuco crystal violet were identified as reaction photoproducts.

The photobleaching of BSA-bound CV⁺also leads to photofragmentation of BSA both in air-equilibrated and nitrogen-purged samples, as assessed through the characterization of high molecular weight photoproducts.¹⁷ The formation of protein photofragmentation products on photolysis of nitrogen-purged samples of CV⁺noncovalently bound to BSA suggests that this TAM⁺dye may show some phototoxicity toward living cells even under conditions of total anaerobiosis. The importance of molecular oxygen with regard to the overall phototoxicity of CV⁺toward living cells was, however, clearly characterized in experiments carried out with L1210 leukemia cells. The phototoxic effect of CV⁺was lowered on purging the cell suspension with argon immediately before irradiation, and enhanced on purging the suspension with oxygen (data not shown). In FIG. 4 the mechanistic step that describes the protection of CV⁺from photodecomposition via quenching of CV⁺triplet by molecular oxygen with the concomitant formation of singlet oxygen represents the primary photochemical event of the classical mechanism of photosensitization type II.^{38,39} Conversely, the oxidation of a semi-reduced CV● radical back to the original dye cation with the concomitant formation of a superoxide radical represents an early step of the classical mechanism of photosensitization type I. Both the quenching of a CV⁺triplet and the oxidation of the semi-reduced dye radical back to its original cation by molecular oxygen represent reaction routes that protect the dye from photo-bleaching. Accordingly, the quantum efficiency of photobleaching of BSA-bound CV⁺is twofold higher in nitrogen-purged samples as compared with air-equilibrated samples. Time-resolved spectroscopic data have indicated that the partial protection against dye photobleaching observed in the presence of oxygen is controlled by the oxidation of the semireduced dye radical back to the original dye cation.^{17,37}

Therefore, one hypothesis is that the phototoxicity of TAM⁺dyes toward living cells must be driven primarily by the mechanisms of photosensitization type I. This hypothesis is additionally validated by the fact that it was impossible to characterize clearly the formation of singlet oxygen during the photolysis of BSA-bound CV⁺using a sensitive chemical method for the detection of singlet oxygen (the p-nitrosodimethylaniline method⁴⁰).³⁷ Interestingly, the test tube findings and the mechanistic hypothesis are in total resonance with those by Docampo and co-workers on the mechanisms of trypanocidal activity of CV⁺. Through a series of elegant experiments involving electron spin resonance spectroscopy and chemical methods for the detection of superoxide radical and singlet oxygen, Docampo and co-workers ^{9,11,12} have demonstrated that the trypanocidal activity of CV⁺occurs primarily by way of the semi-reduced CV⁺radical/superoxide radical route. Further evidence in support of this hypothesis was obtained through the comparison of the effect of ascorbate on CV⁺photobleaching efficiency and phototoxicity toward Trypanospoma cruzi. Ascorbate is a good electron donor that facilitates the formation of the semi-reduced CV●radical on photolysis of CV⁺in the test tube, as indicated by an enhancement in the dye's photobleaching efficiency, and the presence of ascorbate in samples containing T. cruzi is known to increase the efficiency with which CV⁺photoinactivates this blood parasite.^{13,34}

Possible Role of Dye Aggregation on Photosensitization Efficacy:

It has been previously observed that the formation of TAM⁺dimers in aqueous media is facilitated by the protein binding event.³⁷ The formation of dimers of tri-para-substituted TAM⁺dyes is indicated by the appearance of a hypsochromically-shifted absorption band that overlaps with the spectral shoulder of the respective dye monomer, whereas the formation of higher aggregates leads to larger shifts toward the blue region of the spectrum.^{37,41} As predicted by the molecular exciton theory,⁴² a hypsochromic shift in the maximum absorption band after dye dimerization is an indication of the formation of structures in which the transition dipole moments of the constituent dye molecules are parallel to each other (H-type aggregates), and these aggregates are typically more photoreactive than the respective dye monomers.³⁷ Biopolymer-assisted TAM⁺aggregation occurs under conditions of high biopolymer loading (high dye to biopolymer ratios) and is apparently a common phenomenon, since all biopolymers tested so far (hexokinase, human serum albumin, BSA, and DNA) facilitate TAM⁺aggregation (data not shown).

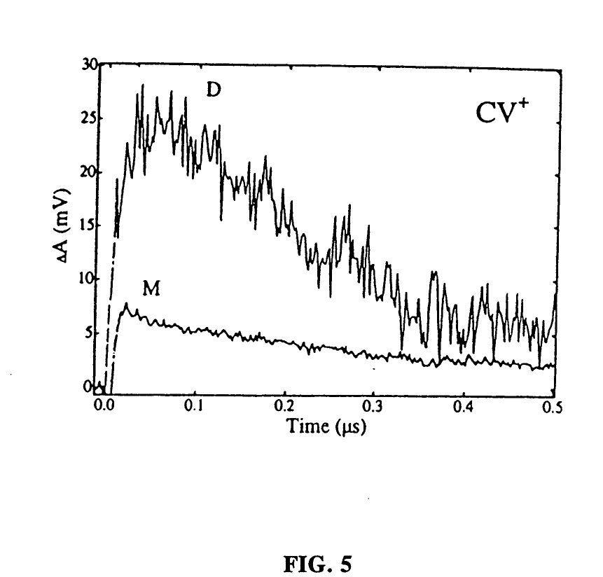

Considering that CV⁺efficiently accumulates in cell mitochondria (FIG. 1), it is reasonable to presume that some dye aggregation may take place in that cellular environment. To explore whether dye aggregation might play a significant role in the efficacy of TAM⁺dyes to induce cell destruction, the extent of formation of reaction transients (triplet species and free radicals) upon photolysis of CV⁺noncovalently bound to BSA both as a monomer and as a dimer was examined. FIG. 5 shows transient decay profiles of 10 μM CV⁺solutions on 560 nm photolysis, as measured at the region of maximum absorbance of the semi-reduced CV● radical (405 nm).⁴³ The formation of a substantial population of reaction transients was observed on photolysis of CV⁺bound to BSA as a monomer, whereas the formation of dimers in the protein environment further increased the formation of transient species. (Compare the amplitude, ΔOD, of the respective transient signals). No transient signal was observed in the absence of protein. The effect of self-association on the photoreactivity of this TAM⁺dye was also examined through the comparison of photobleaching efficiency values. The photobleaching efficiency of BSA-bound CV⁺dimer in air-equilibrated samples (1.1×10⁻⁴) was found to be eightfold higher than the photobleaching efficiency of its monomer (1.3×10⁻⁵).

The enhancement observed in the population of reaction transients and photobleaching efficiency of CV⁺bound to BSA as a dimer compared with its monomeric form indicates that this dye is more likely to engage in photoinduced electron transfer processes when in its dimeric form. Consequently, it is reasonable to infer that the formation of dye aggregates in the mitochondrial environment may play a role on the efficacy of CV⁺and other mitochondrial dyes for photoinduced destruction of living cells. The design of optimized TAM⁺photosensitizers for the effective and selective targeting of tumor cell mitochondria must, therefore, also include considerations on how molecular structure affects dye aggregation.

### CONCLUSIONS

The selective phototoxicity of CV⁺(and closely-related homologs) to tumor cells, along with its mitochondrial nature, indicates that these TAM⁺dyes can be used as novel mitochondria-targeted drugs for a variety of therapeutic applications, most notably the photochemical purging of residual tumor cells from autologous bone marrow grafts with minimum toxicity toward normal cells. The selectivity to which CV⁺and other extensively conjugated cationic compounds mediate the destruction of tumor cells is believed to depend primarily on the lipophilic/hydrophilic character of each specific photosensitizer. Optimized mitochondria-targeted TAM⁺photosensitizers should, therefore, display both high photochemical reactivity and lipophilic/hydrophilic character similar to that of CV⁺or even lower, closer to the lipophilic/hydrophilic character of Rh123. The photochemical data generated so far suggest that CV⁺and other TAM⁺dyes develop their phototoxic effect primarily through the photosensitization mechanisms type I. The data also suggest that protein-assisted TAM⁺aggregation may represent an important factor with regard to the phototoxicity of these compounds in complex biologic environments.

### BIBLIOGRAPHY

1. Chen L B. 1988. Mitochondrial membrane potential in living cells. Ann Rev Cel Biol 4:155-181.

2. Davis S, Weiss M J, Wong J R, Lampidis T J, Chen L B. 1985. Mitochondrial and plasma membrane potentials cause unusual accumulation and retention of rhodamine 123 by human breast adenocarcinoma-derived MCF-7 cells. J Biol Chem 260:13844-13850.

3. Kawakami M, Koya K, Ukai T, Tatsuta N, Ikegawa A, Ogawa K, Shishido T, Chen L B. 1997. Synthesis and evaluation of novel rhodacyanine dyes that exhibit antitumor activity. J Med Chem 40:3151-3160.

4. Modica-Napolitano J S, Brunelli B T, Keizo K, Chen L B. 1998. Photoactivation enhances the mitochondrial toxicity of the cationic rhodacyanine MKT-077. Cancer Res 58:71-75.

5. Koya K, Li Y, Wang H, Ukai T, Tatsuta N, Kawakami M, Shishido T, Chen L B. 1996. MKT-077, a novel rhodacyanine dye in clinical trials, exhibits anticarcinoma activity in preclinical studies based on selective mitochondrial accumulation. Cancer Res 56:538-543.

6. Ara G, Apprille J R. Malis C D, Kane S B, Cincotta L, Foley J, Bonventre J V, Oseroff A R. 1987. Mechanism of mitochondrial photosensitization by the cationic dye, N,N-bis(2-ethyl-1,3-dioxolane)krypto-cyanine (EDKC): preferential inactivation of complex I in the electron transport chain. Cancer Res 47:6580-6585.

7. Morgan J, Whitaker J E, Oseroff A R. 1998. GRP78 induction by calcium ionophore potentiates PDT using the mitochondrial targeting dye Victoria Blue BO. Photochem Photobiol 67:155-164.

8. Nussenzweig V, Sonntag R, Biancalana A, Freitas J L P, Amato Neto V, Kloetzel J. 1953. Action of certain dyes on T. cruzi in vitro. The use of gentian violet to prevent the transmission of Chagas. Hospital (Rio J) 44:731-744.

9. Docampo R, Moreno S N J, Muniz R P A, Mason R P. 1983. Light enhanced free radical formation and trypanocidal action of gentian violet (crystal violet). Science 220:1292-1294.

10. Gadelha F R, Moreno S N J, De Souza W, Cruz FS, Docampo R. 1989. The mitochondrion of Tiypanosoma cruzi is a target of CV toxicity. Molec Biochem Parasitol 34:117-126.

11. Reszka K, Cruz F S, Docampo R. 1986. Photosensitization by the trypanocidal agent crystal violet. Type I versus type II reactions. Chem Biol Interactions 58:161-172.

12. Docampo R, Moreno S N J, Gadeiha F R, De Souza W, Cruz F. 1988. Prevention of Chagas' disease resulting from blood transfusion by treatment of blood: toxicity and mode of action of gentian violet. Biomed Environ Sci 1:406413.

13. Ramirez L E, Lages-Silva E, Pianetti G M, Rab⁻lo RMC, Bordin J O, Mornes-Souza H. 1995. Prevention of transfusion-associated Chagas' disease by sterilization of Trypanosoma cruzi-infected blood with gentian violet, ascorbic acid, and light. Transfusion 35:226-230.

14. Moraes-Souza H, Bordin J O. 1996. Strategies for prevention of transfusion-associated Chagas' disease. Transf Med Rev 10: 161-170.

15. Duxbury D F. 1993. The photochemistry and photophysics of triphenylmethane dyes in solid and liquid media. Chem Rev 93:381-433.

16. Fisher V, Harrelson W G Jr, Chingel C F, Mason R P. 1984. Spectroscopic studies of cutaneous photosensitizing agents. V. Spin trapping and direct electron spin resonance investigations of the photoreduction of gentian (crystal) violet. Photochem Photobiol 7:11-119.

17. Baptista M S, Indig G L. 1998. Effect of BSA binding on photophysical and photochemical properties of triarylmethane dyes.J Phys Chem 2B:4678-4688.

18. Leo A, Hansch C, Elkins D. 1971. Partition coefficients and their uses. Chem Rev 71:525-016.

19. Hatchard C G, Parker CA. 1956. A new sensitive chemical actinometer II. Potassium ferrioxalate as a standard chemical actinometer. Proc R Soc London, Ser A 235:518-536.

20. Hamai 5, Hirayama F. 1983. Actinometric determination of absolute fluorescence quantum yields. J Phys Chem 87:83-89.

21. Liao Y, Bohne C. 1996. Alcohol effect on equilibrium constants and dissociation dynamics of xanthone-cyclodextrin complexes. J Phys Chem 100:734-743.

22. Albota M A, Xu C, Webb W W. 1998: Two-photon fluorescence excitation cross section of biomolecular probes from 690 nm to 960 nm. Appl Optics 37:7352-7356.

23. Anderson G S, Guenther W H H, Searle li, Bilitz J M, Krieg M, Sieber F. 1996. Inactivation of photosensitizing merocyanine dyes by plasma, serum and serum components. Photochem Photobiol 64:683-687.

24. Gaffney D K, Schober S L, Sieber F. 1990. Merocyanine 540-sensitized photoinactivation of leukemia cells: Role of oxygen and effects on plasma membrane integrity and mitochondrial respiration. Exp Hematol 18:23-26.

25. Sieber F, Spivak J L, Sutcliffe A M. 1984. Selective killing of leukemic cells by merocyanine 540-mediated photosensitization. Proc Natl Acad Sci USA 81:7584-7587.

26. Yamazaki T, Sato Y, Sieber F. 1997. Role of cytoprotective mechanisms in the photochemical purging of autologous bone marrow grafts. Exp Hematel 25:629-637.

27. Iscove N N, Sieber F, Winterhalter K H. 1974. Erythroid colony formation in cultures of mouse and human bone marrow: analysis of the requirement for erythropoietin by gel filtration and affinity chromatography on agarose-concanavalin-A. J Cell Physiol 83:309-320.

28. Denk W, Strickler J H, Webb W W. 1990. 2-photon laser scanning fluorescence spectroscopy. Science 248:73-76.

29. Chance B. 1970. Fluorescent probe environment and the structural and charge changes in energy coupling of mitochondrial membranes. Proc Natl Acad Sci USA 67:560-564.

30. Moreno S N J, Gadelha F R, Docampo R. 1988. Crystal violet as an uncoupler of oxidative phosphorylation in rat liver mitochondria. J Biol Chem 263: 12493-12499.

31. Kowaltowski A J, Turin J, Indig G L, Vercesi AE. 1999. Mitochondrial effects of triarylmethane dyes. J Bioenerg Biomembr 31:579-588.

32. Fiedorowicz M, Pituch-Noworolska A, Zembala M. 1997. The effect of victoria blue BO on peripheral blood mononuclear and leukemic cells. Photochem Photobiol 65:855-861.

33. Viola A, Hadjur C, Jeunet A, Julliard M. 1996. Electron paramagnetic resonance evidence of the generation of superoxide and hydroxyl radicals by irradiation of a new photodynamic therapy photo-sensitizer, Victoria Blue B O. J Photechem Photobiol B: Biol 32:49-58.

34. Docampo R, Moreno S N J, Cruz F S. 1988. Enhancement of the cytotoxicity of crystal violet against Trypanospoma cruzi in the blood by ascorbate. Molec Biochem Parasitol 27:241-248.

35. Sundstrom V, Gilibro T, Bergstrom H. 1982. Picosecond kinetics of radiationless relaxations of triphenylmethane dyes. Evidence for a rapid excited-state equilibrium between states of differing geometry. Chem Phys 73:439458.

36. Vogel M, Rettig W. 1985. Efficient intramolecular fluorescence quenching in triphenylmethane dyes involving excited states with charge separation and twisted conformations. Ber Bunsen-Ges Phys Chem 89:962-968.

37. Bartlett J A, Indig G L. 1999. Effect of self-association and protein binding on the photochemical reactivity of triarylmethanes. Implications of non-covalent interactions on the competition between photosensitization mechanisms type I and type II. Photochem Photobiol 70:490-498.

38. Foote C S. 1968. Mechanism of photosensitized oxidation. Science 162:963-970.

39. Foote C S. 1991. Definition of type I and type II photosensitized oxidation. Photochem Photobiol 54:659.

40. Kraljic I, El Moshni S. 1978. A new method for the detection of singlet oxygen in aqueous solution. Photochem Photobiol 28:577-581.

41. Lueck H B, Rice B L, McHale J L. 1992. Aggregation of triphenylmethane dyes in aqueous solution: dimerization and trimerization of crystal violet and ethyl violet. Spectrochim Acta 48A, 819-828.

42. Kasha M, Rawls H R, El-Bayoumi MA. 1965. The exciton model in molecular spectroscopy. Pure Appl Chem 11:371-392.

43. Jockusch S, Turro NJ. 1999. Radical addition rate constants to acrylates and oxygen: α-hydroxy and α-amino radicals produced by photolysis of photoinitiators. J Am Chem Soc 121:3921-3925.

44. Chen, L. B. (1998) Mitochondrial Membrane Potential in Living Cells. Ann. Rev. Cell Biol. 4:155-181.

45. Yamazaki, T. and Sieber, F. (1997) The Alkyl-lysophospholipid, ET-18-OCH₃ Synergistically Enhances the Merocyanine 540 mediated Photoinactivation of Leukemia Cells: Implications for the Extracorporeal Purging of Autologous Hematopoietic Stem Cells, Bone Marrow Transplantation, 19:113-119.

b 46. Yamazaki, T., et al. (1997) Role of Cytoprotective Mechanisms in the Photchemical Purging of Autologous Bone Marrow Grafts, Experimental Hematology 25:629-637.

What is claimed is:

1. A method of purging malignant cells from a mixture containing malignant and non-malignant cells, the method comprising:
(a) contacting the mixture with a compound selected from the group consisting of: 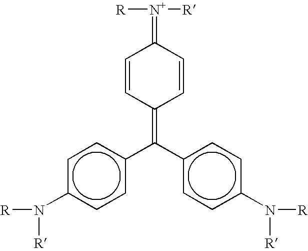
wherein each R and R′ is independently selected from the group consisting of hydrogen and C₁-C₆ linear or branched alkyl;
(b) exposing the mixture from step (a) to radiation of a suitable wavelength to photoactivate the compound, thereby inducing death of malignant cells in the mixture.

2. The method of claim 1, wherein in step (a), the mixture is contacted with a compound wherein each R and R′ are methyl.

3. The method of claim 1, wherein the mixture comprises bone marrow cells.

4. The method of claim 3, wherein the bone marrow cells are cells taken from a patient suffering from leukemia, disseminated multiple myeloma, or lymphoma.

5. The method of claim 3, wherein the bone marrow cells are human bone marrow cells.

6. A method of killing cancer cells or inhibiting growth of cancer cells, in vitro, in vivo, or ex vivo, the method comprising:
(a) contacting the cancer cells with a compound selected from the group consisting of: 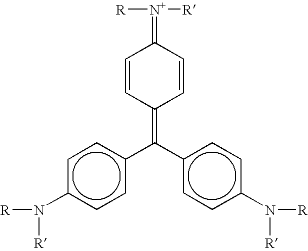
wherein each R and R′ is independently selected from the group consisting of hydrogen and C₁-C₆ linear or branched alkyl;
(b) exposing the cancer cells from step (a) to radiation of a suitable wavelength to photoactivate the compound, whereby cancer cell death or cancer cell growth inhibition results.

7. The method of claim 6, wherein in step (a), the cancer cells are contacted with the compound in vitro.

8. The method of claim 6, wherein in step (a), the cancer cells are contacted with the compound in vivo.

9. The method of claim 6, wherein in step (a), the cancer cells are contacted with the compound ex vivo.

10. The method of claim 6, wherein in step (a), the cancer cells are contacted with a compound wherein each R and R′ is methyl.
